(19) United States
(12) Patent Application Publication
(10) Pub. No.: US 2023/0034514 A1
(43) Pub. Date: Feb. 2, 2023

(54) TISSUE ADHESION COMPOSITION WITH BIO-TISSUE ADHESIVENESS AND BONDING FORCE AND PREPARATION METHOD THEREFOR

(71) Applicant: AJOU UNIVERSITY INDUSTRY-ACADEMIC COOPERATION FOUNDATION, Suwon-si (KR)

(72) Inventor: Byoung-Hyun MIN, Anyang-si (KR)

(73) Assignee: AJOU UNIVERSITY INDUSTRY-ACADEMIC COOPERATION FOUNDATION, Suwon-si (KR)

(21) Appl. No.: 17/765,921

(22) PCT Filed: Sep. 29, 2020
(86) PCT No.: PCT/KR2020/013386

§ 371 Date: Oct. 17, 2022

(30) Foreign Application Priority Data
Oct. 2, 2019 (KR) 10-2019-0122142
Sep. 18, 2020 (KR) 10-2020-0120854

(51) Int. Cl. A61L 24/00; C07K 14/47; A61L 27/36; A61L 27/38; A61L 27/44; C12N 5/00

(52) U.S. Cl. CPC A61L 24/00; C07K 14/47; A61L 27/36; A61L 27/3817; A61L 27/3834; A61L 27/44; C12N 5/0068; A61L 27/24

(57) ABSTRACT

The present invention relates to a tissue adhesion agent having improved bio-tissue adhesiveness and bonding force by utilizing an adhesion-related gene. More specifically, a cartilage tissue adhesion composition prepared from fetal cartilage tissue-derived stem cells in which VCAN, CTGF, or EXT1 is inserted and expressed in an upregulated manner was found to show a remarkably superb adhesive force, compared to that prepared from fetal cartilage tissue-derived stem cells in which none of the genes are inserted. Accordingly, the cell composition in which the expression of VCAN, CTGF, or EXT1 is upregulated can be prepared into a tissue adhesion composition having improved bio-tissue adhesiveness and bonding force and VCAN, CTGF, or EXT1 can be provided as an additive composition for a tissue adhesion agent.

### TECHNICAL FIELD

The present disclosure relates to a tissue adhesion agent with increased bio-tissue adhesiveness and bonding force using cells with expression of adhesion-related genes increased.

### BACKGROUND ART

Bioadhesives and sealants are used to seal tissues during surgery or applied therefor and also used as an antihemorrhagic agent and a blocking agent for body fluid and blood, such that biocompatibility is required since they come into contact with the skin. In addition, there should be no toxicity and harmfulness to the living body while not interfering with the healing of the living body.

Medical adhesive materials that are currently in practical use include cyanoacrylate-based, fibrin glue-based, gelatin glue-based, and polyurethane-based materials. Closure Medical of the United States has commercialized an octylcyanoacrylate-based medical tissue adhesive called Dermabond, and it was approved for sale by the European Community in August 1997 and by the US FDA in 1998.

However, the cyanoacrylate-based adhesive has a problem in that it interferes with wound healing since a solidified material is less flexible and hard. In addition, it easily becomes a foreign substance after being encapsulated because it hardly decomposes in vivo. Fibrin glue has a very low adhesive force so that a produced fibrin mass may fall from tissues and is also susceptible to viral infection due to being a blood formulation.

In addition, in the case of a gelatin glue, a bio-derived tissue adhesive treated with gelatin-resorcinol-formalin was developed, and tissue adhesives such as gelatin-glutaraldehyde were also developed. However, formalin or glutaraldehyde used as a crosslinking agent undergoes a cross-linking reaction with proteins in the living body, causing tissue toxicity.

In order to overcome the issues, there is a need for development of a bio-tissue adhesive exhibiting excellent biocompatibility, superior mechanical strength, and fast, strong adhesive force in the presence of moisture.

### DISCLOSURE

### Technical Problem

The present disclosure provides an adhesion-related gene having tissue adhesive force and binding force, and the gene may be used for the development of an adhesive for regenerative treatment of tissues or organs damaged after tissue injury or organ surgery.

### Technical Solutions

Example embodiments of the present disclosure provide a tissue adhesion composition with increased bio-tissue adhesiveness and bonding force, including, as an active ingredient, cells with increased expression of one or more genes selected from the group consisting of CTGF, EXT1, NDST1, ITGA3, CYTL1, TFGB1, SOCS3, VCAN, CHI3L2, and KRT19.

Example embodiments of the present disclosure provide an additive composition for a tissue adhesion agent, including one or more genes selected from the group consisting of CTGF, EXT1, NDST1, ITGA3, CYTL1, TFGB1, SOCS3, VCAN, CHI3L2, and KRT19 as an active ingredient.

Example embodiments of the present disclosure provide a tissue adhesion agent preparation method including inserting one or more genes from the group consisting of CTGF, EXT1, NDST1, ITGA3, CYTL1, TFGB1, SOCS3, VCAN, CHI3L2, and KRT19 into cells, performing primary culture of the gene-inserted cells in a medium containing a cartilage differentiation medium for 5 to 10 days, performing secondary monolayer culture of the primarily cultured cells, and obtaining cell membranes on which the secondarily monolayer cultured cells and extracellular matrix are combined.

### Advantageous Effects

According to example embodiments of the present disclosure, it was confirmed that a cartilage adhesion composition prepared with fetal cartilage derived progenitor cells having increased expression through insertion of VCAN, CTGF, or EXT1 showed remarkably excellent adhesion strength compared to a cartilage adhesion composition prepared with fetal cartilage derived progenitor cells without insertion of the genes. A cell composition in which the expression of VCAN, CTGF, or EXT1 is increased may be prepared as a tissue adhesion composition with increased bio-tissue adhesiveness and bonding force, and VCAN, CTGF, or EXT1 may be provided as an additive composition for a tissue adhesion agent.

### BRIEF DESCRIPTIONS OF THE DRAWINGS

FIG. 1 shows results of a microarray analysis comparing an adhesion control mechanism-related gene expressed in a sheet-cultured adhesive using human fetal cartilage derived cells and an adhesion control mechanism-related gene expressed in the sheet-cultured adhesive using human adult cartilage derived cells.

FIG. 2 shows results of a microarray analysis comparing an adhesion control mechanism-related gene expressed in a sheet-cultured adhesive using human fetal cartilage derived cells and an adhesion-related genes expressed when human adult cartilage derived cells are cultured by a general cell culture method.

FIG. 3 shows results of a microarray analysis comparing an adhesion control mechanism-related gene expressed in a sheet-cultured adhesive using human fetal cartilage derived cells and an adhesion-related gene expressed when human bone marrow derived mesenchymal stem cells are cultured by a general cell culture method.

FIG. 4 shows results of DNA cloning after making CTGF, EXT1, or VCAN, which are target genes related to adhesion, bound to a pCMV-HA vector, respectively.

FIG. 5 shows images of results of insertion of a target gene into human cartilage cells (chondrocytes) and culturing the same under the same condition.

FIG. 6 shows results of identifying the gene expression level via real time polymerase chain reaction (RT-PCR) after insertion of a target gene into human cartilage cells(chondrocyte).

FIG. 7 shows images showing results of insertion of a target gene into fetal cartilage derived progenitor cells and culturing the same under the same condition.

FIG. 8 shows a result of real time polymerase chain reaction (RT-PCR) after insertion of a target gene into fetal cartilage derived progenitor cells.

FIG. 9 shows images of results of insertion of siRNA of a target gene into fetal cartilage derived progenitor cells of an example embodiment of the present disclosure and culturing the same under the same conditions.

FIG. 10 shows results of identifying the gene expression level by performing real time polymerase chain reaction (RT-PCR) after insertion of siRNA of a target gene into fetal cartilage derived progenitor cells of an example embodiment of the present disclosure and culturing the same.

FIG. 11 is a process of identifying adhesion strength of a cartilage adhesion composition (TAP-C) prepared with fetal cartilage derived progenitor cells.

FIG. 12 shows results of identifying adhesion strength of a cartilage adhesion composition prepared with human cartilage cells(chondrocyte) and fetal cartilage derived progenitor cells (FCPC), respectively, into which CTGF or EXT1 gene is inserted or not.

FIG. 13 shows a result of identifying the tissue adhesiveness of a bio-tissue conjugate prepared using VCAN which is an adhesion-related gene.

### BEST MODE

Hereinafter, the present disclosure will be described in more detail.

An example embodiment of the present disclosure is to identify an adhesion control mechanism-related gene whose expression is increased in a tissue adhesive which contains human fetal cartilage derived cells and fetal cartilage derived extracellular matrix as an active ingredient and is in the form of a gel-type cell sheet, and also to provide a conjugate for bonding damaged tissues using the same.

An example embodiment of the present disclosure may provide a tissue adhesion composition which includes cells with increased expression of one or more genes selected from the group consisting of CTGF, EXT1, NDST1, ITGA3, CYTL1, TFGB1, SOCS3, VCAN, CHI3L2, and KRT19, and exhibits increased bio-tissue adhesiveness and bonding force.

The cell may be selected from the group consisting of human cartilage cells (chondrocytes) and human fetal cartilage derived progenitor cells.

The tissue adhesion composition may be used as a tissue adhesive, a tissue suture agent, an anti-adhesion agent, a hemostatic agent, or a wound dressing agent.

An example embodiment of the present disclosure may provide an additive composition for a tissue adhesion agent, including one or more genes selected from the group consisting of CTGF, EXT1, NDST1, ITGA3, CYTL1, TFGB1, SOCS3, VCAN, CHI3L2, and KRT19 as an active ingredient.

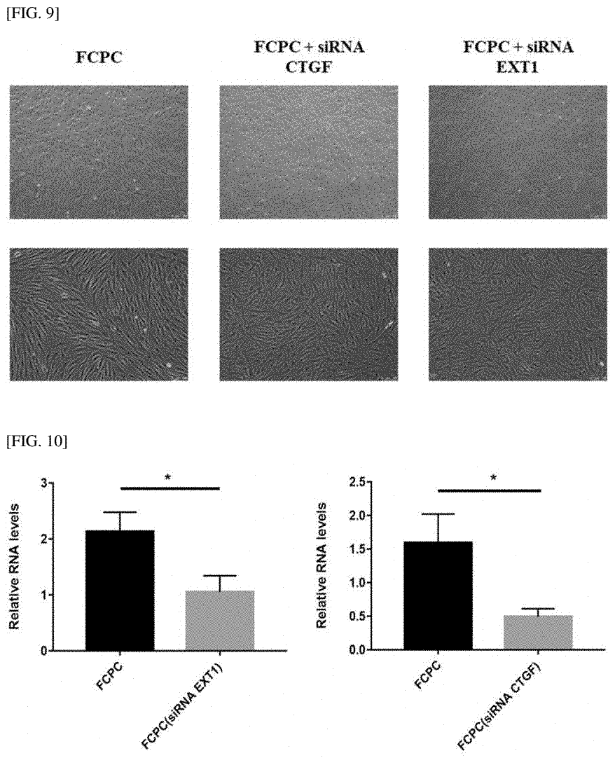

According to an example embodiment of the present disclosure, the tissue adhesion composition (TAP), prepared with human fetal cartilage derived progenitor cells whose expression was increased through insertion of VCAN, CTGF, or EXT1 gene, was inserted into a cartilage damaged model made from donated cartilages of a patient. A jig with a diameter of 5 mm was brought into contact with the inserted TAP for attachment, and the resistance was checked by pulling at a speed of 1.3 mm/min until the jig was separated from the TAP. As a result of comparison with the cartilage adhesion composition (TAP-C) prepared using the fetal cartilage derived progenitor cells, though no clear difference was observed in the first week and the second week of culture as shown in FIG. 9, remarkably superior adhesion strength was observed in the gene-inserted group in the third week of culture compared to the TAP-C group. It was confirmed that the adhesion strength of the cartilage adhesion composition prepared with cells having a gene knocked down through siRNA insertion was significantly reduced compared to the TAP-C group.

From the results above, a cell composition with increased expression of CTGF, EXT1, NDST1, ITGA3, CYTL1, TFGB1, SOCS3, VCAN, CHI3L2, and KRT19 may be provided as a tissue adhesion composition with increased bio-tissue adhesiveness and bonding force, and the genes may be provided as an additive composition for a tissue adhesion agent.

In addition, an example embodiment of the present disclosure may provide a tissue adhesion agent preparation method which includes inserting one or more genes from the group consisting of CTGF, EXT1, NDST1, ITGA3, CYTL1, TFGB1, SOCS3, VCAN, CHI3L2, and KRT19 into cells, performing primary culture of the gene-inserted cells in a medium containing a cartilage differentiation medium for 5 to 10 days, performing secondary monolayer culture using the primarily cultured cells, and obtaining cell membranes on which the secondarily monolayer cultured cells and extracellular matrix are combined.

The cells may be selected from the group consisting of human cartilage cells(chondrocyte) and human fetal cartilage derived progenitor cells.

The secondary monolayer culture of the primarily cultured cells may be monolayer culture of the primarily cultured cells in a growth medium for 15 to 20 days.

The cell membrane includes a cell with increased expression of one or more genes selected from the group consisting of CTGF, EXT1, NDST1, ITGA3, CYTL1, TFGB1, SOCS3, VCAN, CHI3L2, and KRT19 and exhibits increased tissue adhesiveness and bonding force.

The term “adhesion agent” as used herein refers to a drug that promotes adhesion after skin or muscle tissue is healed.

### MODES FOR CARRYING OUT INVENTION

Hereinafter, examples will be described in detail to help the understanding of the present disclosure. However, the following examples are merely illustrative of the content of the present disclosure, and the scope of the present disclosure is not limited to the following examples. The examples of the present disclosure are provided to more completely explain the present disclosure to those skilled in the art.

<Example 1> Isolation and culture of human cartilage cells (chondrocyte)

After cartilages isolated from knee joint were washed with phosphate buffered saline (PBS), a Dulbecco's Modified Eagle Medium (DMEM, Gibco, Grand Island, NY) containing 0.2% (w/v) collagenase (Worthington Biochemical Corp., Lakewood, NJ) was added, followed by culture in an incubator in the presence of 5% CO₂ at 37° C. for 4 hours. Cartilage derived progenitor cells released after complete digestion of cartilages were centrifuged at 1700 rpm for 10 minutes to obtain precipitated cartilage cells(chondrocyte), and seeded in a tissue culture dish [150 mm(dia.)×20 mm(h)] at a cell density of 1×10⁶.

<Example 2> Isolation and culture of human fetal cartilage derived progenitor cells (FCPCs)

Cartilage tissue derived progenior cells were isolated from the knee joint of a 12 to 15-week-old fetus (Source: IRB NO. AJIRB-CRO-07-139 approved by the Ethics Committee of Ajou University Hospital). Briefly, after cartilages isolated from the knee joint were washed with phosphate buffered saline (PBS), a Dulbecco's Modified Eagle Medium (DMEM, Gibco, Grand Island, NY) containing 0.2% (w/v) collagenase (Worthington Biochemical Corp., Lakewood, NJ) was added, followed by culture in an incubator in the presence of 5% CO₂ at 37° C. for 4 hours. Cartilage tissue derived progenitor cells released after complete digestion of cartilages were centrifuged at 1700 rpm for 10 minutes to obtain precipitated fetal cartilage derived progenitor cells (FCPCs) and seeded in a tissue culture dish [150 mm(dia.)×20 mm(h)] at a cell density of 1×10⁶.

<Example 3> Preparation of a composition for tissue adhesion (TAP) having tissue adhesion and differentiation properties

After diluting cartilage cells(chondrocyte) obtained in Example 1 and 2 to a cell density of 1×10⁶, culture was performed on 6 wells in an incubator in the presence of 5% CO₂ at 37° C. for a week using a medium containing a cartilage cells(chondrocyte) differentiation medium (1% antibiotic-antimycotic), 1.0 mg/mL of insulin, 0.55 mg/mL of human transferrin, 0.5 mg/mL of sodium selenite, 50 μg/mL of ascorbic acid, 1.25 mg/mL of bovine serum albumin (BSA), 100 nM of dexamethasone, 40 μg/mL of proline, and DMEM-HG added with 10 ng/ml of TGF-β, followed by monolayer culture in a DMEM medium added with 10% fetal bovine serum (FBS), 50 units/mL of penicillin, and 50 μg/mL of streptomycin for 15 to 18 days. After the culture, the medium was removed, and PBS was added to obtain cell membranes in the form of a sheet combined with extracellular matrix.

<Example 4> Microarray analysis

Comparison was made with a gene expression profile of TAP prepared in Example 3. Whole human genome Agilent 4×44K Oligonucleotide Microarray (Macrogen, Korea) was used to select genes with expression remarkably increased in the two TAPs prepared in Example 3.

Briefly, total RNA was extracted from TAP, human cartilage cells(chondrocyte), or bone marrow stromal cells and applied to microarray chip hybridization. To compare the gene expression profiles of healthy human cartilage cells(chondrocyte) and TAP, representative genes for histocompatibility were selected, and microarray analysis was conducted.

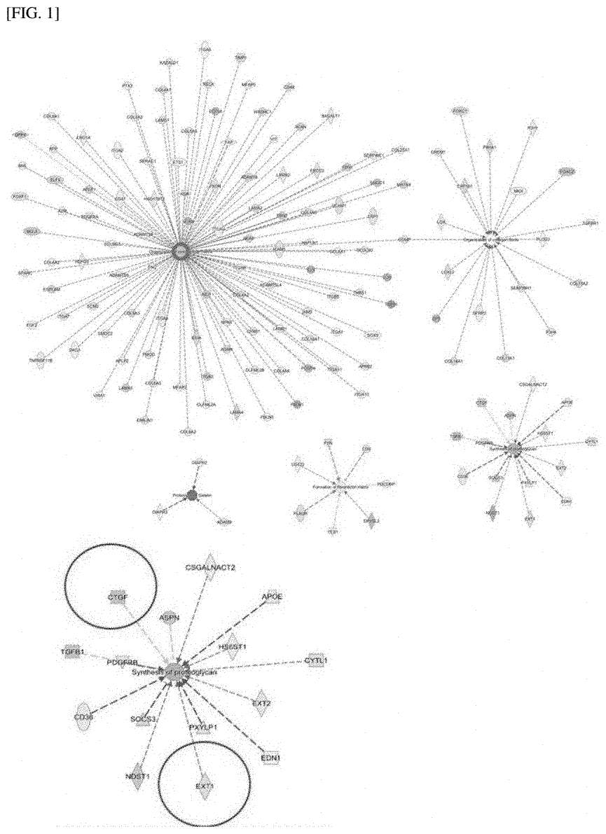

Thereafter, the gene expression was detected as shown in FIG. 1 through comparison between a gene expressed when human fetal cartilage derived cells are cultured in a sheet with excellent bioadhesiveness and an adhesion-related genes expressed when human adult cartilage derived cells are cultured in a sheet.

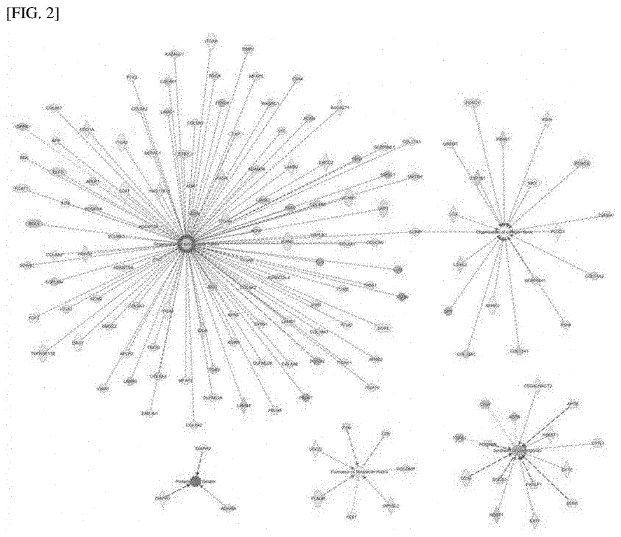

In addition, as a result of comparing the expression of the gene expressed when human fetal cartilage derived cells are cultured in the sheet with excellent bioadhesiveness and the adhesion-related gene expressed when human adult cartilage derived cells are cultured by a general cell culture method, the gene expression was detected as shown in FIG. 2

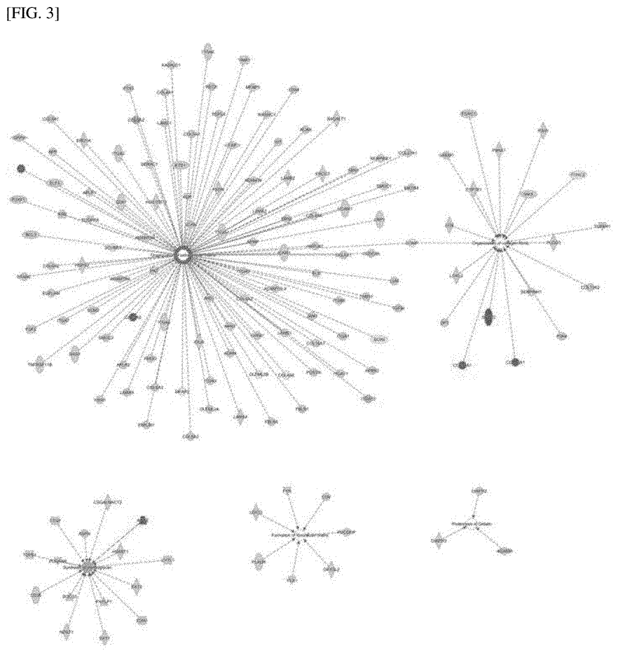

Finally, by comparing the expression of a gene expressed when human fetal cartilage derived cells are cultured in the sheet with excellent bioadhesiveness and an adhesion-related genes expressed when human bone marrow derived mesenchymal stem cells are cultured by a general cell culture method, the gene expression was detected as shown in FIG. 3. From the above results, detection of the genes expressed when human fetal cartilage derived cells were cultured in the form of a sheet enabled identification of specifically expressed adhesion-related genes, as shown in TABLE 1 below.

TABLE 1

| Gene | | Gene No. Sequence |
|---|---|---|
| CTGF | CCN2 or connective tissue | HM015591 |
| | growth factor | |
| NDST1 | Bifunctional heparan sulfate | U17970 |
| | N-deacetylase/N-sulfotransferase 1 | |
| EXT1 | Exostosin-1 | Q16394 |
| ITGA3 | Integrin alpha-3 | AC002401 |
| CYTL1 | Cytokine-like protein 1 | BC031391 |
| TGFB1 | Transforming growth factor | BT007245 |
| | beta-1 proprotein | |
| SOCS3 | Suppressor of cytokine signaling 3 | AF159854 |
| VCAN | Versican | U26555 |
| CHI3L2 | Chitinase-3-like protein 2 | U49835 |
| KRT19 | Keratin, type I cytoskeletal 19 | AF202321 |

<Example 5> Preparation of a composition for tissue adhesion (TAP) having tissue adhesion and differentiation properties involving target gene treatment

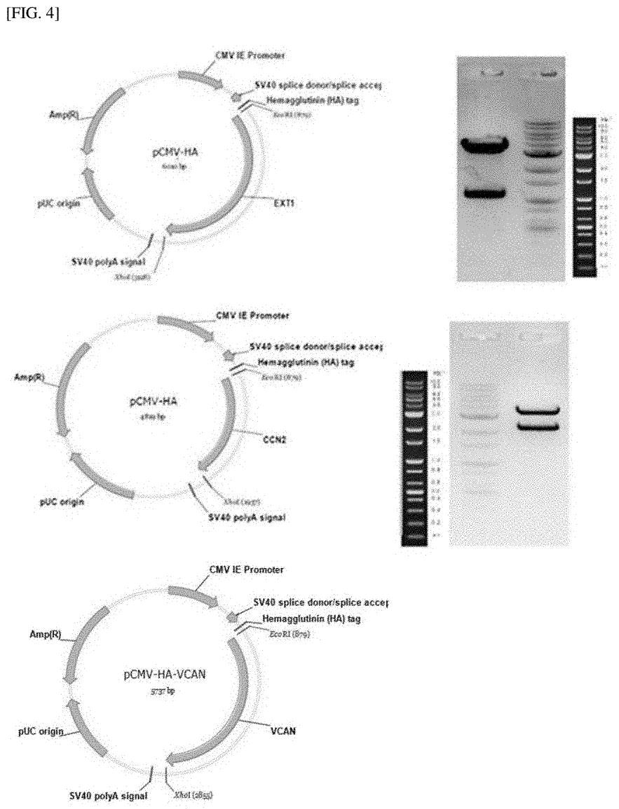

After combining CTGF (CCN2) and EXT1 identified in Example 4 with a vector as shown in FIG. 4, an insert gene was prepared through cloning. Gene knock-in was performed using Gene Transfection Reagent (Lipofectamine 3000 Thermo). A gene knock-down experiment was performed by treating siRNA (Bionics) of each gene under the same conditions.

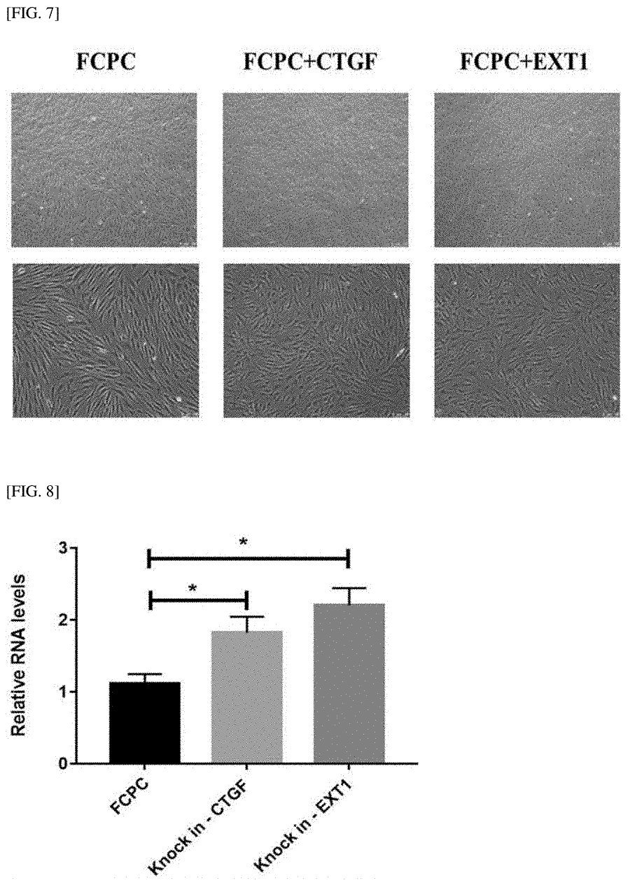

As shown in FIG. 5 and FIG. 7, human cartilage cells(chondrocyte) and fetal cartilage derived progenitor cells (FCPC) were treated with the inserted gene for 3 days to insert the gene into the cells, and cell membranes were obtained by the same method as in Example 3.

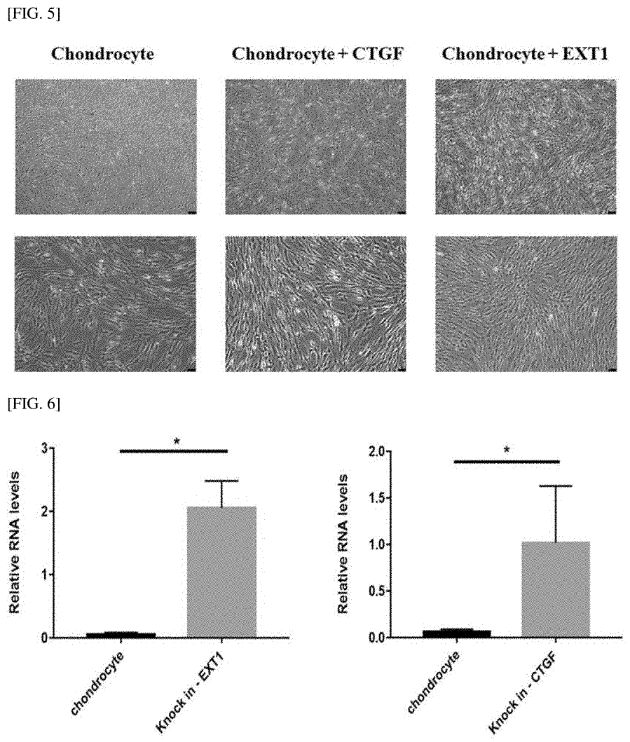

In order to detect gene expression in the composition for tissue adhesion prepared by the above process, real time polymerase chain reaction (RT-PCR) was performed using cell membranes obtained from cartilage cells(chondrocyte) and fetal cartilage derived progenitor cells (FCPC) cultured by treating each gene. As a result, it was confirmed that the expression of EXT1 and CTGF was increased in the cartilage cells(chondrocyte) into which the target gene was inserted as shown in FIG. 6, and it was also confirmed that the expression of EXT1 and CTGF was increased even in fetal cartilage derived progenitor cells into which the target gene was inserted as shown in FIG. 8. On the other hand, as shown in FIG. 7, it was confirmed that the expression of EXT1 and CTGF was suppressed in fetal cartilage derived progenitor cells into which the siRNA of the target gene was inserted.

From the above results, it was confirmed that EXT1 and CTGF were effectively inserted into cartilage cells(chondrocyte) and fetal cartilage derived progenitor cells, and the cell membrane with increased expression of EXT1 and CTGF genes was obtained.

<Example 6> Confirmation of adhesion strength of composition for tissue adhesion

Adhesive force was compared to confirm the adhesion strength of the composition for tissue adhesion treated with TAP prepared in Examples 3 and 5 above and each gene.

First, the patient's cartilage to be discarded after joint arthroplasty was donated with a consent form. Prepared was a model with cartilage damaged on the surface of the donated patient's cartilages using a 6 mm biopsy punch, and the prepared composition for adhesion was inserted.

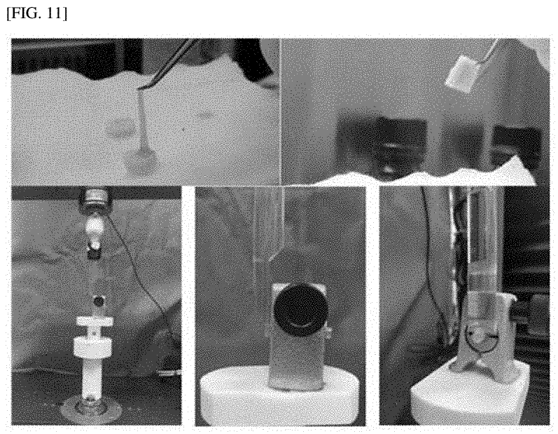

Next, after binding and attachment to the TAP into which the jig with a diameter of 5 mm was inserted by the same process as FIG. 11, the resistance was checked until the jig was separated from the TAP by pulling at a speed of 1.3 mm/min.

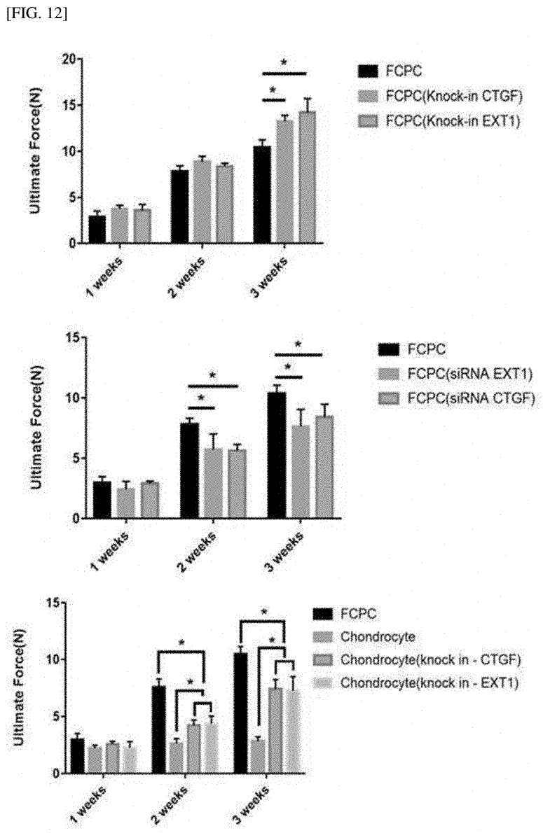

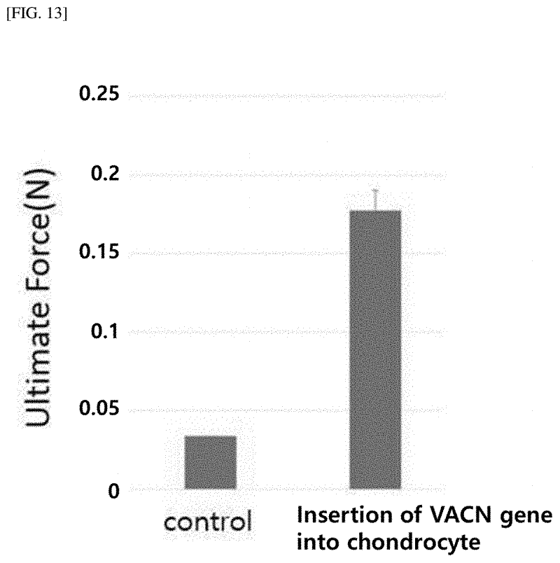

As a result of comparing the cartilage adhesion composition (TAP-C) prepared by using fetal cartilage derived progenitor cells and the cell membranes treated with each gene, though no clear difference was observed between the first week and the second week of culture as shown in FIG. 12, it was confirmed that the adhesion strength was remarkably excellent in the gene insertion group compared to the TAP-C group at the third week of culture. It was also confirmed that the adhesion strength of the cartilage adhesion composition prepared with knock-down cells through siRNA insertion was significantly reduced compared to the TAP-C group.

In addition, as a result of checking adhesiveness after insertion of VCAN into cartilage cells(chondrocyte) by the same method and preparation of a bio-tissue adhesive, it was confirmed that the cartilage-bone adhesive force of the bio-tissue adhesive was increased as shown in FIG. 13.

Hereinafter, examples will be described in detail to help the understanding of the present disclosure. However, the following examples are merely illustrative of the content of the present disclosure, and the scope of the present disclosure is not limited to the following examples. The examples of the present disclosure are provided to more completely explain the present disclosure to those skilled in the art.

1-4. (canceled)

5. A method of preparing a tissue adhesion agent comprising:
inserting one or more genes selected from the group consisting of CTGF, EXT1, NDST1, ITGA3, CYTL1, TFGB1, SOCS3, VCAN, CHI3L2, and KRT19 into a cell;
performing primary culture of the cell, into which the one or more genes are inserted, in a medium containing comprising a cartilage differentiation medium for 5 to 10 days;
performing secondary monolayer culture of the cell primarily cultured; and
obtaining a cell membrane, on which the cell secondarily cultured and extracellular matrix are combined.

6. The method of claim 5, wherein the cell is selected from the group consisting of a human cartilage cell (chondrocyte) and a human fetal cartilage derived progenitor cell.

7. The method of claim 5, wherein performing secondary monolayer culture of the cell comprises performing monolayer culture of the cell primarily cultured in a growth medium for 15 to 20 days.

8. The method of claim 5, wherein the cell membrane comprises a cell with increased expression of one or more genes selected from the group consisting of CTGF, EXT1, NDST1, ITGA3, CYTL1, TFGB1, SOCS3, VCAN, CHI3L2, and KRT19 and exhibits increased tissue adhesiveness and bonding force.

9. A method of adhering two or more tissues each other comprising treating the two or more tissues with a tissue adhesion composition of comprising a cell with increased expression of one or more genes selected from the group consisting of CTGF, EXT1, NDST1, ITGA3, CYTL1, TFGB1, SOCS3, VCAN, CHI3L2, and KRT19 as an active ingredient.

10. The method of claim 9, wherein the cell is selected from the group consisting of a human cartilage cell (chondrocyte) and a human fetal cartilage derived progenitor cell.

11. The method of claim 9, wherein the tissue adhesion composition is used as a tissue adhesive, a tissue suture agent, an anti-adhesion agent, a hemostatic agent, or a wound dressing agent.

12. A method of adhering two or more tissues each other comprising treating the two or more tissues with an additive composition for a tissue adhesion agent comprising one or more genes selected from the group consisting of CTGF, EXT1, NDST1, ITGA3, CYTL1, TFGB1, SOCS3, VCAN, CHI3L2, and KRT19 as an active ingredient.

13. A method of adhering two or more tissues each other comprising treating the two or more tissues with the tissue adhesion agent prepared by the method of claim 5.
